(19) United States
(12) Patent Application Publication
(10) Pub. No.: US 2025/0332113 A1
(43) Pub. Date: Oct. 30, 2025

(54) ADHESIVE PARTICLES FOR ACTIVE DELIVERY

(71) Applicant: THE TRUSTEES OF THE UNIVERSITY OF PENNSYLVANIA, Philadelphia, PA (US)

(72) Inventors: Daeyeon LEE, Wynnewood, PA (US); Minjun OH, Philadelphia, PA (US); Jaehyun KIM, Philadelphia, PA (US); Li-Wei CHANG, Rocky Hill, CT (US); Robert Leon MAUCK, Philadelphia, PA (US); George R. DODGE, Philadelphia, PA (US); Hyun KOO, Philadelphia, PA (US)

(21) Appl. No.: 18/868,259

(22) PCT Filed: Jun. 1, 2023
(86) PCT No.: PCT/US2023/067752

§ 371 Date: Nov. 22, 2024

Related U.S. Application Data
(60) Provisional application No. 63/365,700, filed on Jun. 1, 2022.

(51) Int. Cl. A61K 9/50; A61K 9/00

(52) U.S. Cl. CPC A61K 9/5031; A61K 9/006

(57) ABSTRACT

Provided are adhesive dendritic particles, which particles can be used for sustained delivery of an active agent to a location or locations at which the particles are adhered. Also provided are related methods of use and methods of formulating the disclosed particles.

### CROSS-REFERENCE TO RELATED APPLICATIONS

The present application claims priority to and the benefit of U.S. patent application No. 63/365,700, “Adhesive Particles For Active Delivery” (filed Jun. 1, 2022). All foregoing applications are incorporated herein by reference in their entireties for any and all purposes.

### GOVERNMENT RIGHTS

This invention was made with government support under AR071340awarded by the National Institutes of Health. The government has certain rights in the invention.

### TECHNICAL FIELD

The present disclosure relates to the field of emulsions and the field of dendritic particles.

### BACKGROUND

Existing particle-based drug delivery systems are not well-suited to prolonged delivery of an active agent to a given area, as such existing particles typically circulate freely throughout the body and do not remain in their desired location for any appreciable amount of time. Accordingly, there is a long-felt need in the art for systems and methods for persistent delivery of active agents to desired body locations.

### SUMMARY

In meeting the described long-felt needs, the present disclosure provides an adhesive composition, comprising: an adhesive dendritic particle that comprises (1) a polymeric phase with dendrites extending therefrom, and (2) at least one active ingredient, and (a) the adhesive dendritic particle having an aqueous phase disposed within the polymeric phase and at least one active ingredient present within the aqueous phase, (b) the adhesive dendritic particle having at least one active ingredient present in the polymeric phase, or (c) both (a) and (b), and the adhesive dendritic particle being configured to adhere to a location of a subject so as to allow for release of at least one active ingredient to the location.

Also provided is a method, comprising administering a composition according to the present disclosure (e.g., according to any one of Aspects 1-16) to a subject such that the adhesive dendritic particle adheres to a location of the subject so as to allow for release of at least one active ingredient from the adhesive dendritic particle at the location.

Further provided is a method, comprising: forming an emulsion comprising an adhesive dendritic particle that comprises (1) a polymeric phase with dendrites extending therefrom, and (2) at least one active ingredient, and (a) the adhesive dendritic particle having an aqueous phase disposed within the polymeric phase and at least one active ingredient being present within the aqueous phase, or (b) the adhesive dendritic particle having at least one active ingredient being present in the polymeric phase, or (c) both (a) and (b).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1A-1B. Microparticle adhesion test on extracted natural teeth. (FIG. 1A) Extracted incisor teeth. (FIG. 1B) Fluorescent images show adhesion properties of microparticles depending on the structure of microparticles. Hairy particles show strong adhesion on the natural teeth even after 3 washes. (Inset: scanning electron microscope image of a hairy microparticle.)

FIGS. 2A-2B. Simulated drug releasing profile from PEG-PLGA double emulsions-based hairy microparticles using two different fluorescent molecules. (FIG. 2A) Loaded Alexa Fluor® 647 (1025.2 g/mol) and (FIG. 2B) GFP (27 kDa) were released depending on the time and detected in supernatant.

FIG. 3 provides a depiction of using the disclosed compositions to deliver an anti-biofilm agent or agents to a biofilm that has formed on a subject's tooth.

FIG. 4 provides an illustration of the adhesion effected between two glass slides (measured by the number of United States quarter coins needed to break the adhesion between the slides) for various illustrative adhesive dendritic particles according to the present disclosure.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

### Illustrative Disclosure

As a non-limiting illustration of the disclosed adhesive drug delivery system, adhesive microcapsules were prepared using interfacial instability and self-assembly of biocompatible amphiphilic PEG-b-PLGA block copolymers (BCPs) in an aqueous solution. The rapid solvent (oil) evaporation of water-in-oil-in-water (W/O/W) emulsion induces both spontaneous increasement in interfacial area and self-assembly of BCPs, resulting in the formation of hairy microcapsules.

To fabricate hairy microcapsules, i) two-step sequential emulsification and ii) microfluidic flow-focusing method were used to prepare double-emulsion droplets. We use 2 wt % poly (vinyl alcohol) (PVA, Mw: 8.9k) aqueous solution with water soluble drugs (As proof of concept, green fluorescent protein (GFP) and Alexa Fluor 647 were used) in the innermost phase. For the middle phase, 50 mg/ml PEG (5k)-b-PLGA (20k) has been dissolved in dichloromethane (DCM) by vortex and sonication. As the continuous phase, we use 2 wt % PVA aqueous solution to stabilize double emulsions.

For the production of multiple double emulsions through two-step sequential emulsification, the most inner phase (aqueous phase) and middle phase (oil phase) are emulsified at volume ratio 1:10 (inner phase: middle phase) using vortex mixing and sonication. At this step, we observe a water-in-oil (W/O) single emulsion. To prepare the double emulsion, continuous phase (aqueous phase) and single emulsion are mixed as 20:1 volume ratio using a homogenizer (IKA T18 basic, IKA, Germany). Depends on the volume ratio, the particle size can be controlled. Lastly, generated double emulsions are collected into a glass petri dish to evaporate DCM (oil phase) with magnetic stirring at 250 rpm and air ventilation system for the rapid evaporation. During the evaporation of DCM, PEG-b-PLGA microcapsules form hairy shaped microcapsules.

For the production of multiple double emulsions through microfluidic generation method, we use a microfluidic device that combines co-flow and flow focusing geometry with typical dimensions of orifice for inner fluid and collection were 20˜100 and 100˜200 μm, respectively. The distance between two capillaries for inner fluid and collection was adjusted to be 20˜150 μm. The flow rates of the inner aqueous, middle oil, and outer aqueous phases are precisely controlled for generating desired size and shape of double emulsions.

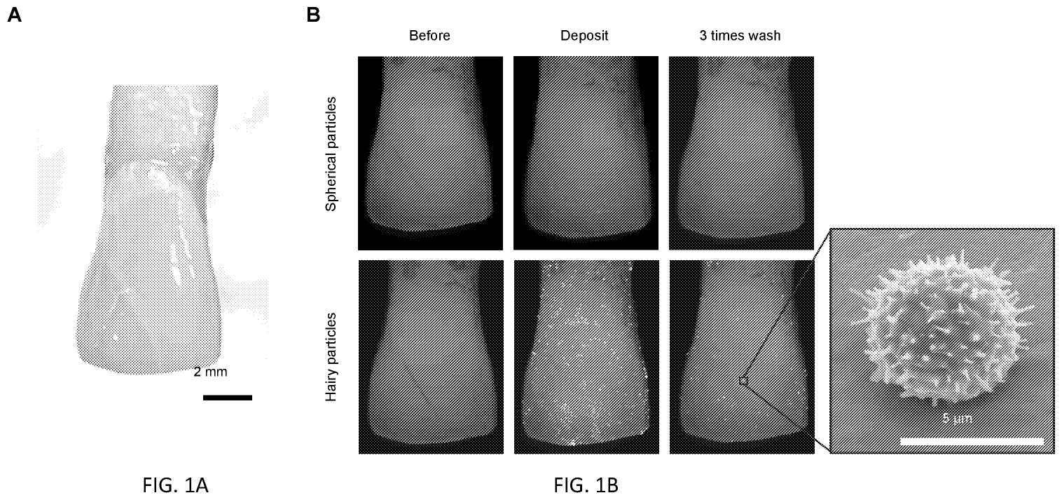

The adhesive properties of microparticles are tested and compared. FIGS. 1A-1B show natural teeth and attached spherical and hairy particles with fluorescent dye (Nile Red, in DCM). The spherical particles are not easily attached to the natural teeth and attached particles are easily washed out. However, the hairy particles show better adhesion than spherical particles. Also, even after 3 times washing, attached hairy particles are easily observed on natural teeth surface.

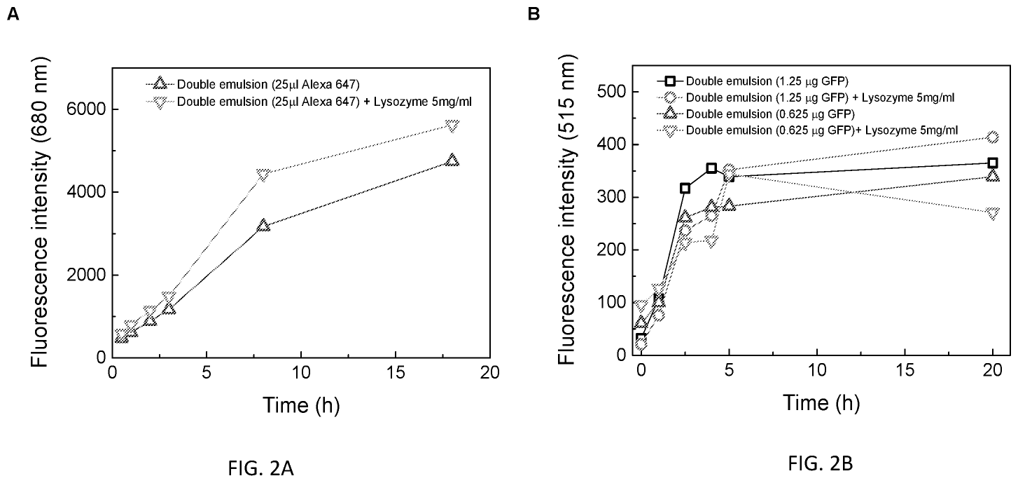

To test drug encapsulating and releasing properties, we use two different sizes of fluorescent molecules (Alexa Fluor® 647, 1025.2 g/mol and GFP, 27 kDa). We load these molecules into most inner phase and generate double emulsions through two-step sequential emulsification. After fully drying of DCM, hairy microparticles are collected and washed 3 times by centrifugation at 3000 rcf for 1 min with deionized water. To determine the release profile, we place a sample in 0.1 M PBS buffer (pH=7.4, 5 ml total) at 37° C. and monitor the release of the fluorescent molecules. The fluorescent intensity in supernatant are measured using a BioTek plate reader. FIGS. 2A-2B show the release profile of both Alexa Fluor 647 and GFP. Based on the fluorescent intensity of end point, about 22-26% of Alexa Fluor 647 and 19-35% of GFP are released (compared to the amount injected into the emulsions) from microcapsules.

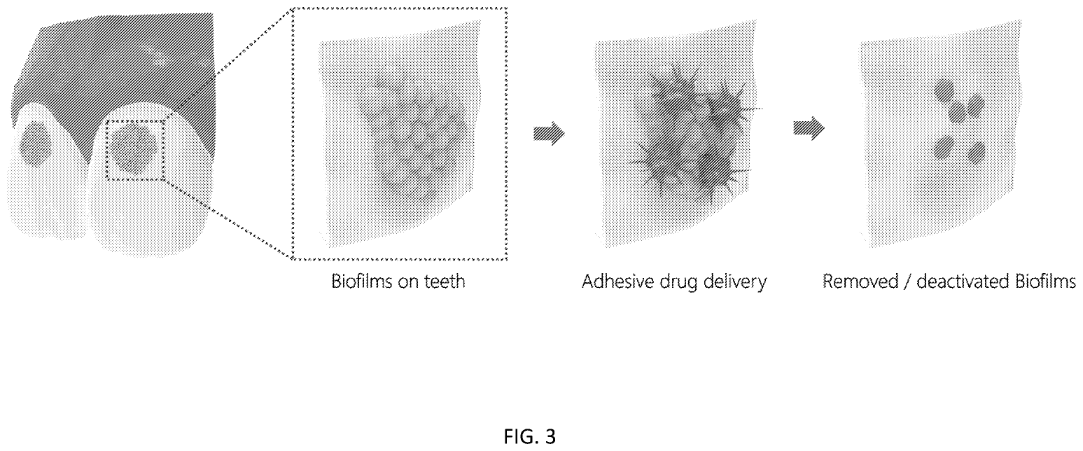

FIG. 3 provides a depiction of using the disclosed compositions to deliver an anti-biofilm agent or agents to a biofilm that has formed on a subject's tooth.

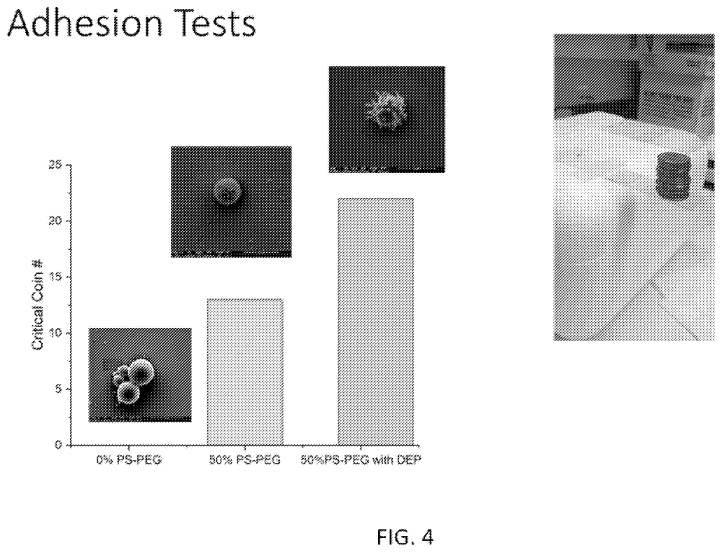

FIG. 4 provides an illustration of the adhesion effected between two glass slides (measured by the number of United States quarter coins needed to break the adhesion between the slides) for various illustrative adhesive dendritic particles according to the present disclosure.

### Emulsion Formation via Homogenizer

The following provides a non-limiting illustration of forming the disclosed emulsions using a homogenizer-based approach.

### PEG (5k)-b-PLGA (20k) Block Copolymer Particle Fabrication Method (Single Emulsion)

(8.9k) PVA 2 wt. % Aqueous solution has been prepared as medium. Magnetic stirred at 700 rpm, heating over Tg of PVA.

PEG (5k)-b-PLGA (20k) was dissolved in Dichloromethane (DCM), 50 mg/1ml. Vortex and sonication were used for mixing.

PVA aqueous solution and DCM mixture were mixed to make single emulsion. The mixing ratio is 20:1 (PVA aqueous solution: DCM-polymer mixture)

IKA homogenizer T18 was used, mixed at 10k RPM for 1 minute.

To evaporate the DCM at room temperature, a glass petri dish was used as container. The glass petri was positioned on a magnetic stirrer and mixed at 250 RPM.

DCM was then evaporated with air ventilation system with the magnetic stirring.

### PEG (5k)-b-PLGA (20k) Block Copolymer Particle Fabrication Method (Double Emulsion)

(8.9k) PVA 2wt. % Aqueous solution was prepared as the continuous phase. Magnetic stirred at 700 rpm, heating above the Tg of PVA.

PEG (5k)-b-PLGA (20k) has been dissolved in Dichloromethane (DCM), 50 mg/1 ml. Vortex and sonication used for mixing. This mixture was prepared as the middle phase.

(8.9k) PVA 2 wt. %, GFP (or ALEXA) 1.25 mg/1 ml (or 1 mg˜50 mg/1 ml) mixture prepared as the inner phase. The mixture volume ratio is 9:1 (PVA aqueous solution: GFP aqueous solution).

The inner phase and middle phase were mixed at a volume ratio 1:10 (inner phase: middle phase). Vortex and sonication were used, and a single emulsion observed at this step.

To prepare the double emulsion, the continuous phase and single emulsion phase were mixed in a 20:1 volume ratio. Depending on the volume ratio, the particle size can be controlled.

IKA homogenizer T18 used to mix at 10k RPM for 1 minute.

To evaporate the DCM at room temperature, a glass petri dish was used as a container. The glass petri was positioned on the magnetic stirrer and mixed at 250 RPM.

DCM evaporated with air ventilation system with the magnetic stirring.

### Aspects

The following Aspects are illustrative only and do not limit the scope of the present disclosure or the appended claims.

Aspect 1. An adhesive composition, comprising:

an adhesive dendritic particle that comprises (1) a polymeric phase with dendrites extending therefrom, and (2) at least one active ingredient, and

(a) the adhesive dendritic particle having an aqueous phase disposed within the polymeric phase and at least one active ingredient present within the aqueous phase,

(b) the adhesive dendritic particle having at least one active ingredient present in the polymeric phase, or

(c) both (a) and (b), and

the adhesive dendritic particle being configured to adhere to a location of a subject so as to allow for release of at least one active ingredient to the location.

Aspect 2. The composition of Aspect 1, wherein the polymeric phase comprises an amphiphilic block copolymer, which can be an amphiphilic diblock copolymer. This is not a requirement, as the polymeric phase can comprise other copolymers besides amphiphilic block copolymers.

Aspect 3. The composition of Aspect 2, wherein the amphiphilic diblock copolymer comprises at least one of a poly (lactic-co-glycolic acid) (PLGA)—polyethylene glycol (PEG) diblock copolymer, a polystyrene (PS)—polyethylene glycol (PEG) diblock copolymer, and a polylactic acid (PLA)—polyethylene glycol (PEG) diblock copolymer.

Aspect 4. The composition of any one of Aspects 1-3, wherein the polymeric phase further comprises a homopolymer, the homopolymer optionally comprising a domain of the amphiphilic diblock copolymer.

Aspect 5. The composition of Aspect 4, wherein the homopolymer comprises a homopolymer of a domain of the amphiphilic block copolymer. As but one example, a composition can include a PLGA-PEG diblock copolymer and a PLGA homopolymer.

Aspect 6. The composition of Aspect 5, wherein the homopolymer comprises a PLGA homopolymer.

Aspect 7. The composition of any one of Aspects 1-6, wherein the adhesive dendritic particle has an aqueous phase disposed within the polymeric phase and at least one active ingredient being present within the aqueous phase.

Aspect 8. The composition of Aspect 7, wherein the adhesive dendritic particle is disposed in an aqueous carrier so as to define a water-in-oil-in-water (W/O/W) emulsion.

Aspect 9. The composition of any one of Aspects 1-8, wherein the active ingredient is dispersed in the polymeric phase.

Aspect 10. The composition of Aspect 9, wherein the adhesive dendritic particle is disposed in an aqueous carrier so as to define an oil-in-water (O/W) emulsion.

Aspect 11. The composition of any one of Aspects 1-10, wherein the polymeric phase defines a shell having a cross-sectional dimension in the range of from about 1 to about 1000 micrometers. The cross-sectional dimension can be, for example, from about 1 to about 1000 micrometers, from about 10 to about 750 micrometers, from about 25 to about 600 micrometers, from about 50 to about 500 micrometers, from about 75 to about 400 micrometers, or from about 100 to about 300 micrometers.

Aspect 12. The composition of Aspect 11, wherein the polymeric phase defines a shell having a cross-sectional dimension in the range of, e.g., from about 5 to about 1000 micrometers.

Aspect 13. The composition of any one of Aspects 1-12, wherein a dendrite has a length in the range of, e.g., from 1 to about 1000 micrometers. A dendrite can have a length of, for example, about 1 to about 1000 micrometers, about 2 to about 750 micrometers, about 5 to about 500 micrometers, about 7 to about 400 micrometers, or even about 10 to about 50 micrometers.

Aspect 14. The composition of Aspect 13, wherein a dendrite has a length in the range of from 1 to about 50 micrometers, optionally in the range of from about 1 to about 10 micrometers, or even about 1 to about 5 micrometers.

Aspect 15. The composition of any one of Aspects 1-14, wherein an active ingredient comprises any one or more of an enzyme, a peptide, a small-molecule drug, a chondrogenic factor, an anabolic compound, a transforming growth factor, a fibroblast growth factor, a connective tissue growth factor, an insulin-like growth factor, a bone morphogenetic protein, an anti-catabolic compound, an anti-inflammatory compound, an antimicrobial (e.g., an antibacterial, and antifungal, and/or an antiviral), antibiofilm compound, an anti-cell death compound.

Aspect 16. The composition of Aspect 15, wherein the active ingredient comprises at least one of an enzyme and a small-molecule drug.

Aspect 17. A method, comprising administering a composition according to any one of Aspects 1-16 to a subject such that the adhesive dendritic particle adheres to a location of the subject so as to allow for release of at least one active ingredient from the adhesive dendritic particle at the location. The administration can be performed, e.g., to treat a medical condition of the subject.

Aspect 18. The method of Aspect 17, wherein the administering is performed under such conditions to effect adhesion between the adhesive dendritic particle and a tooth surface, a gingival surface, a mucosal surface or a biofilm of the subject, the biofilm optionally being an oral biofilm.

Aspect 19. The method of Aspect 17, wherein the administering comprises is performed under such conditions to effect adhesion between the adhesive dendritic particle and a bone, skin, tendon, ligament, cartilage, or vascular tissue of the subject.

Aspect 20. The method of any one of Aspects 17-19, wherein the administering the composition effects adhesion between the adhesive dendritic particle and the subject such that a motion of the subject effects release of the at least one active ingredient from the adhesive dendritic particle.

Aspect 21. The method of Aspect 20, wherein the motion is a physiologic motion. It should be understood that release of an active ingredient from an adhesive dendritic particle can also be effected by, e.g., a change in temperature, a change in pH, administration of an agent that encourages release of the active ingredient from the adhesive dendritic particle, a change in temperature, an externally-administered stimulus (e.g., sonication, vibration, electrical field) and the like.

Aspect 22. A method, comprising:

forming an emulsion comprising an adhesive dendritic particle that comprises (1) a polymeric phase with dendrites extending therefrom, and (2) at least one active ingredient, and

(a) the adhesive dendritic particle having an aqueous phase disposed within the polymeric phase and at least one active ingredient being present within the aqueous phase, or

(b) the adhesive dendritic particle having at least one active ingredient being present in the polymeric phase, or

(c) both (a) and (b).

Aspect 23. The method of Aspect 22, wherein the dendritic particle has an aqueous phase disposed within the polymeric phase and at least one active ingredient present within the aqueous phase and wherein the emulsion is a water-in-oil-in-water double emulsion.

Aspect 24. The method of Aspect 22, wherein at least one active ingredient is present in the polymeric phase and the emulsion is an oil-in-water single emulsion.

Aspect 25. The method of any one of Aspects 22-24, wherein the polymeric phase comprises an amphiphilic block copolymer.

Aspect 26. The method of Aspect 25, wherein the amphiphilic diblock copolymer comprises at least one of a poly (lactic-co-glycolic acid) (PLGA)—polyethylene glycol (PEG) diblock copolymer, a polystyrene (PS)—polyethylene glycol (PEG) diblock copolymer, and a polylactic acid (PLA)—polyethylene glycol (PEG) diblock copolymer.

Aspect 27. The method of Aspect 26, wherein the amphiphilic diblock copolymer comprises at least a poly (lactic-co-glycolic acid) (PLGA)—polyethylene glycol (PEG) diblock copolymer.

Aspect 28. The method of any one of Aspects 22-27, wherein the polymeric phase further comprises a homopolymer, the homopolymer optionally comprising a domain of the amphiphilic diblock copolymer.

Aspect 29. The method of Aspect 28, wherein the homopolymer comprises a homopolymer of a domain of the amphiphilic block copolymer.

Aspect 30. The method of Aspect 29, wherein the homopolymer comprises a PLGA homopolymer.

Aspect 31. The method of any one of Aspects 22-30, wherein forming the emulsion comprises one or more of sonication, homogenization, stirring, or using droplet-forming flow channels.

Aspect 32. The method of Aspect 31, wherein forming the emulsion comprises using droplet-forming flow channels.

Aspect 33. The method of Aspect 32, wherein forming the emulsion comprises wherein forming the emulsion comprises one or more of sonication, homogenization, and stirring.

Aspect 34. The method of any one of Aspects 22-33, further comprising removing a solvent, the solvent optionally being a solvent in which the polymer phase is miscible.

Aspect 35. The method of Aspect 34, wherein the removing comprises one or more of heating, stirring, and ventilating.

1. An adhesive composition, comprising:
an adhesive dendritic particle that comprises (1) a polymeric phase with dendrites extending therefrom, and (2) at least one active ingredient, and
(a) the adhesive dendritic particle having an aqueous phase disposed within the polymeric phase and at least one active ingredient present within the aqueous phase,
(b) the adhesive dendritic particle having at least one active ingredient present in the polymeric phase, or
(c) both (a) and (b), and
the adhesive dendritic particle being configured to adhere to a location of a subject so as to allow for release of at least one active ingredient to the location.

2. The composition of claim 1, wherein the polymeric phase comprises an amphiphilic diblock copolymer.

3. The composition of claim 2, wherein the amphiphilic diblock copolymer comprises at least one of a poly (lactic-co-glycolic acid) (PLGA)—polyethylene glycol (PEG) diblock copolymer, a polystyrene (PS)—polyethylene glycol (PEG) diblock copolymer, and a polylactic acid (PLA)—polyethylene glycol (PEG) diblock copolymer.

4. The composition of claim 2, wherein the polymeric phase further comprises a homopolymer, the homopolymer optionally comprising a domain of the amphiphilic diblock copolymer.

5. The composition of claim 4, wherein the homopolymer comprises a homopolymer of a domain of the amphiphilic diblock copolymer.

6. The composition of claim 5, wherein the homopolymer comprises a PLGA homopolymer.

7. The composition of claim 1, wherein the adhesive dendritic particle has an aqueous phase disposed within the polymeric phase and at least one active ingredient being present within the aqueous phase.

8. The composition of claim 7, wherein the adhesive dendritic particle is disposed in an aqueous carrier so as to define a water-in-oil-in-water (W/O/W) emulsion.

9. The composition of claim 1, wherein the active ingredient is dispersed in the polymeric phase, optionally wherein the adhesive dendritic particle is disposed in an aqueous carrier so as to define an oil-in-water (O/W) emulsion.

10. (canceled)

11. The composition of claim 1, wherein the polymeric phase defines a shell having a cross-sectional dimension in the range of from about 1 to about 1000 micrometers.

12. (canceled)

13. The composition of claim 1, wherein a dendrite has a length in the range of from 1 to about 1000 micrometers, optionally from 1 to about 50 micrometers.

14. (canceled)

15. The composition of claim 1, wherein an active ingredient comprises any one or more of an enzyme, a peptide, a small-molecule drug, a chondrogenic factor, an anabolic compound, a transforming growth factor, a fibroblast growth factor, a connective tissue growth factor, an insulin-like growth factor, a bone morphogenetic protein, an anti-catabolic compound, an anti-inflammatory compound, an antimicrobial (encompassing antibacterial, antifungal, antiviral), antibiofilm compound, an anti-cell death compound, an enzyme. or a small-molecule drug.

16. (canceled)

17. A method, comprising administering a composition according to claim 1 to a subject such that the adhesive dendritic particle adheres to a location of the subject so as to allow for release of at least one active ingredient from the adhesive dendritic particle at the location.

18. The method of claim 17, wherein the administering is performed under such conditions to effect adhesion between the adhesive dendritic particle and a tooth surface, a gingival surface, a mucosal surface or a biofilm of the subject, the biofilm optionally being an oral biofilm.

19. The method of claim 17, wherein the administering comprises is performed under such conditions to effect adhesion between the adhesive dendritic particle and a bone, skin, tendon, ligament, cartilage, or vascular tissue of the subject.

20. The method of claim 17, wherein the administering the composition effects adhesion between the adhesive dendritic particle and the subject such that a motion of the subject effects release of the at least one active ingredient from the adhesive dendritic particle.

21. (canceled)

22. A method, comprising:
forming an emulsion comprising an adhesive dendritic particle that comprises (1) a polymeric phase with dendrites extending therefrom, and (2) at least one active ingredient, and
(a) the adhesive dendritic particle having an aqueous phase disposed within the polymeric phase and at least one active ingredient being present within the aqueous phase, or
(b) the adhesive dendritic particle having at least one active ingredient being present in the polymeric phase, or
(c) both (a) and (b),
optionally wherein forming the emulsion comprises one or more of sonication, homogenization, stirring, or using droplet-forming flow channels.

23. The method of claim 22, wherein (a) the dendritic particle has an aqueous phase disposed within the polymeric phase and at least one active ingredient present within the aqueous phase and wherein the emulsion is a water-in-oil-in-water double emulsion, (b) wherein at least one active ingredient is present in the polymeric phase and the emulsion is an oil-in-water single emulsion, or both (a) and (b).

24. (canceled)

25. The method of claim 22, wherein the polymeric phase comprises an amphiphilic diblock copolymer, the amphiphilic diblock copolymer optionally comprising comprises at least one of a poly (lactic-co-glycolic acid) (PLGA)—polyethylene glycol (PEG) diblock copolymer, a polystyrene (PS)—polyethylene glycol (PEG) diblock copolymer, and a polylactic acid (PLA)—polyethylene glycol (PEG) diblock copolymer.

26. (canceled)

27. (canceled)

28. The method of claim 22, wherein the polymeric phase further comprises a homopolymer, the homopolymer optionally comprising a domain of the amphiphilic diblock copolymer.

29. (canceled)

30. (canceled)

31. (canceled)

32. (canceled)

33. (canceled)

34. (canceled)

35. (canceled)
